(19) United States
(12) Patent Application Publication
(10) Pub. No.: US 2022/0126074 A1
(43) Pub. Date: Apr. 28, 2022

(54) ENCAPSULATION DEVICES AND METHODS OF USE

(71) Applicants: Thomas Kidder Roseberry, Eugene, OR (US); Timothy Gardner, Eugene, OR (US); Jean-Charles Neel, San Francisco, CA (US)

(72) Inventors: Thomas Kidder Roseberry, Eugene, OR (US); Timothy Gardner, Eugene, OR (US); Jean-Charles Neel, San Francisco, CA (US)

(73) Assignee: University of Oregon, Eugene, OR (US)

(21) Appl. No.: 17/508,776

(22) Filed: Oct. 22, 2021

Related U.S. Application Data
(60) Provisional application No. 63/105,084, filed on Oct. 23, 2020.

(51) Int. Cl. A61M 31/00; A61M 5/14

(52) U.S. Cl. CPC A61M 31/00; A61M 5/14

(57) ABSTRACT

Encapsulation devices are provided that comprise an outer wall defining an inner volume, the outer wall comprising a plurality of pores and a plurality of vasculature holes; and a plurality of channels traversing the inner volume, wherein each channel extends from one of the plurality of vasculature holes to another of the plurality of vasculature holes. The device is used for the implantation or delivery of cells, proteins, or other therapeutic molecules. Also provided are methods of treating a disorder in a subject using an encapsulation device described herein. The disclosed encapsulation devices have a porous surface and an internal network of channels passing through the device and connecting with the outside of the device.

### CROSS REFERENCE TO RELATED APPLICATION

This application claims the benefit of U.S. Provisional Application No. 63/105,084, filed Oct. 23, 2020, which is incorporated herein by reference in its entirety.

### FIELD

This disclosure relates to encapsulation devices, for example, implantable cell encapsulation devices, and methods of their use, such as for medical and therapeutic purposes.

### BACKGROUND

The treatment of several diseases may include the implantation of functional cells that can secrete a biological factor that a patient requires. For example, diabetic patients can be assisted by the implantation of insulin-secreting cells such as pancreatic islet cells. Existing cell delivery methods may have limited therapeutic efficacy due to the implanted cells not vascularizing sufficiently following implantation. In the absence of vascularization, the tissue-implanted cells may lack adequate nutrient access, which can lead to their gradual disappearance and a decrease in their capability of releasing adequate levels of therapeutic agent. Further, the implanted cells may necrose under stress factors. Implanted cells may also become ineffective or die due to immune attack on the implanted cells. Finally, implanted cells can escape into the body, risking the formation of tumors, such as in the case of induced pluripotent stem cell-derived implanted cells.

### SUMMARY

Disclosed herein are encapsulation devices, such as for implantation or delivery of cells, peptides, proteins, exosomes, or other therapeutic molecules, and methods of their use, such as for treating a disorder in a subject, or for providing beneficial substances or biologics to the subject. The disclosed encapsulation devices have a plurality of small pores on the outer surface and an internal network of channels passing through the device and connecting with the outside of the device. The channels encourage infiltration by endothelial cells for blood vessel formation and provide high surface area for vascular formation. The sub-cellular sized small pores allow molecules or exosomes to pass between the inside and outside of the device, while keeping the contents of the device from spreading to the outer environment and protecting the contents from detection or attack by the immune system. The encapsulation devices disclosed herein address concerns with previous methods of implanting cells, including cell nutrient supply, cell protection from immune system, and protection of the body against the implanted cells.

The disclosure includes embodiments of an encapsulation device that comprise an outer wall defining an inner volume, the outer wall comprising a plurality of pores and a plurality of vasculature holes; and a plurality of channels traversing the inner volume, wherein each channel extends from one of the plurality of vasculature holes to another of the plurality of vasculature holes. In some examples, each channel extends from the one of the plurality of vasculature holes on a surface of the outer wall to another of the plurality of vasculature holes on a different (e.g., an opposing or adjacent) surface of the outer wall. In some examples, the outer wall further comprises at least one port selectively coupled to an inner lumen of the inner volume, for example for introducing a payload to the inner volume. In some examples, the port further comprises a closure mechanism. In some examples, the device further comprises a payload in the inner volume of the device. In some examples, the device further comprises an attachment mechanism for attaching the device to an anatomical structure, such as a blood vessel or nerve. In other examples, the device further comprises a tether, such as a thin film.

The disclosure also includes methods of treating a condition or disorder in a subject including implanting a disclosed encapsulation device including a payload into a cavity of the subject. In some examples, the cavity is a peritoneal cavity of the subject. In some examples, the methods further include inserting a payload in the inner volume of the disclosed encapsulation device, for example, injecting the payload into the inner volume of the encapsulation device through the injection port.

The foregoing and other features of the disclosure will become more apparent from the following detailed description, which proceeds with reference to the accompanying figures.

### BRIEF DESCRIPTION OF THE DRAWINGS

FIG. 1 is an isometric view of an embodiment of an encapsulation device of the disclosure.

FIG. 2 is a cross-sectional view of an embodiment of an encapsulation device of the disclosure.

FIG. 3 shows a top perspective view of an embodiment of an encapsulation device of the disclosure displaying an injection port on a top wall of the device.

FIG. 4 shows a top perspective view of an embodiment of an encapsulation device of the disclosure with a trap door covering the injection port in a closed position.

FIG. 5 shows a top perspective view of an embodiment of an encapsulation device of the disclosure with a trap door covering the injection port in an open position.

FIG. 6 shows a cross sectional view of an embodiment of an encapsulation device of the disclosure displaying communication of the injection port with an inner lumen of the device.

FIG. 7A shows an embodiment of a spherical encapsulation device.

FIG. 7B shows a cross-sectional view of the embodiment of FIG. 7A.

FIG. 8A shows an embodiment of a dumbbell shaped encapsulation device.

FIG. 8B shows a cross-sectional view of the embodiment of FIG. 8A.

FIG. 9 shows an embodiment of a dome shaped encapsulation device anchored to an implantable thin film substrate.

FIG. 10A shows an embodiment of an encapsulation device with an attachment mechanism for attaching the device to an anatomical structure.

FIG. 10B shows a cross-sectional view of the embodiment of FIG. 10A.

FIG. 10C shows the embodiment of FIG. 10A attached to an anatomical structure.

FIG. 11 shows the embodiment of FIG. 1 configured with non-vascular structures on an external surface.

FIG. 12 shows an embodiment of multiple encapsulation devices positioned relative to one another and a substrate via the non-vascular structures of FIG. 11.

### DETAILED DESCRIPTION

The encapsulation device disclosed herein provides a semi-permeable enclosure for placement in the body of a subject in order to treat various disorders or conditions or to provide additional benefits. The porosity of each surface of the device, along with a cage-like inner structure, which in some examples includes a fractal geometry to maximize surface area, allows for high efficiency vascularization of cells seeded into a lumen of the device. The structure can be custom designed for a given application (e.g., for a target area of treatment or implantation) and can be fabricated using 3D printing, reducing manufacturing costs and allowing for simplicity and flexibility.

It will be appreciated that while the encapsulation devices and methods described herein are discussed primarily with respect to cellular payloads, this is not meant to be limiting and the devices and methods are also suitable for release of drugs (such as time-released drugs), proteins or peptides, or other therapeutic molecules and/or biologics.

Unless otherwise explained, all technical and scientific terms used herein have the same meaning as commonly understood by one of ordinary skill in the art to which the encapsulation device belongs. The singular terms “a,” “an,” and “the” include plural referents unless the context clearly indicates otherwise. Although materials and methods similar or equivalent to those described herein can be used in the practice or testing of this disclosure, suitable methods and materials are described below. The term “comprises” means “includes.” All publications, patent applications, patents, and other references mentioned herein are incorporated by reference in their entirety for all purposes. In case of conflict, the present specification, including explanation of terms, will control. In addition, the materials, methods, and examples are illustrative only and not intended to be limiting.

I. Overview of Several Embodiments

Disclosed herein are embodiments of an encapsulation device. In some embodiments, the encapsulation device comprises an outer wall defining an inner volume, the outer wall comprising a plurality of pores (e.g., nanopores or micropores) and a plurality of vasculature holes; and a plurality of channels traversing the inner volume, wherein each channel extends from one of the plurality of vasculature holes to another of the plurality of vasculature holes. In some examples, the plurality of pores comprise openings of less than about 2 μm in diameter. In some examples, the plurality of vasculature holes comprise holes of at least two distinct diameters. In some examples, the plurality of vasculature holes comprise holes of about 10 μm to about 1 mm in diameter.

In some examples, each channel extends from the one of the plurality of vasculature holes on a surface of the outer wall to another of the plurality of vasculature holes on an opposing surface of the outer wall. In some examples, the opposing surface is a diametrically opposing surface. In some examples, each channel extends from the one of the plurality of vasculature holes on one surface of the outer wall to another of the plurality of vasculature holes on another surface of the outer wall (such as an adjacent or orthogonal surface of the outer wall). In some examples, the plurality of channels include a first channel extending from a vasculature hole of a larger diameter to another vasculature hole of the same diameter, and a second channel extending from a vasculature hole of a smaller diameter to another vasculature hole of the same diameter. In some examples, the first channel is orthogonal to the second channel within the inner volume. In some examples, a diameter of a given channel matches a diameter of a corresponding vasculature hole that the given channel extends to or from. In other examples, the inner volume includes a network of interconnected channels. In further examples, each of the channels comprise the plurality of pores. In some examples, the inner volume is divided into an inner lumen and an outer lumen by the plurality of channels, the inner lumen comprising a region of the inner volume external to the channels, the outer lumen comprising a region of the inner volume internal to the channels. The channels may be straight, curved, or a combination thereof. In some examples, a channel may not directly extend from a first vasculature hole to a second vasculature hole. For example, a channel may extend from a first vasculature hole and connect to a second channel in the device, where the second channel extends between two different vasculature holes (such as a “T” shaped connection).

In some examples, non-vascular structures (e.g., solid columns, beams, struts, wall thickness changes, scaffolds, etc.) can be printed inside or outside of the lumen to provide structural stability. In some examples, the non-vascular structures can be positioned (such as indexing pins or holes for indexing pins) so as to align vasculature holes of a first device with vasculature holes of a second device to promote vascularized connections between the first device and the second device. In some examples, a first set of non-vascular structures may be printed on a surface of an outer wall of a first device and a second set of non-vascular structures may be printed on a surface of an outer wall of a second device, the first set of non-vascular structure configured to connect with (e.g., a mated connection) the second set of non-vascular structures, thereby coupling the first device to the second device to promote vascularized connections between the first device and the second device.

In some examples, the outer wall further comprises at least one port selectively coupled to the inner lumen of the inner volume. In some examples, the at least one port comprises a diameter or width of about 100 μm. In some examples, the port further comprises a closure mechanism. In some examples, the closure mechanism is a trap door or a flap.

In some examples, the device is made of a polymer. In some examples, the polymer is selected from any one of methacrylated alginate, poly-(ethylene glycol) diacrylate, 2-(Hydroxymethyl)-2-[[(1-oxoallyl)oxy]methyl]-1,3-propanediyl-diacrylate, a hybrid organic-inorganic resin such as Ormocomp®, SU-8, cellulose, collagen, chitosan, gelatin methacrylate, SZ2080, and my also include a photoinitiator (such as Irgacure®). In some examples, the polymer is doped with small micro-scale or nano-scale solid particles such as silica, carbon nanotubes, or other ceramics, or metals. In some examples, the device further comprises a payload in the inner volume of the device. In some examples, the payload is in the inner lumen of the inner volume. In further examples, the payload comprises a plurality of cells, proteins, peptides, nucleic acids, exosomes, or small molecule therapeutics.

The disclosure also includes embodiments of a method of treating a condition or disorder in a subject including implanting a disclosed encapsulation device including a payload in the inner volume into a cavity of the subject. In some examples, vascularization of the encapsulation device through the plurality of channels occurs following the implanting. In some examples, the payload comprises a plurality of cells, proteins, peptides, nucleic acids, exosomes, or small molecule therapeutics. Also disclosed are methods of inserting a payload in the inner volume of the disclosed encapsulation device. In some examples, the payload is injected into the inner volume of the encapsulation device through the injection port.

The disclosure also includes embodiments of a method of manufacturing the disclosed encapsulation device. Porous materials used in currently available encapsulation strategies use probabilistic pore sizes. This means that the pore size is a range, and some pores end up large enough to allow immune cells to infiltrate through the surface. Using microfabrication techniques as disclosed herein, near uniform hole sizes throughout the surface of the device (such as nanopores) can be created that are small enough to protect the contents of the device from immune detection.

Currently known strategies for manufacture of enclosures rely on the enclosures being made of two surfaces welded together in a pouch configuration. Still other known strategies have been geometrically constrained by manufacturing processes. In the encapsulation devices described herein, the methods of manufacturing the devices disclosed herein, as well as the methods of treating a disorder using the devices, geometric flexibility is provided through the use of fractal geometries that allow cells to remain close to a surface of the device, or close to blood vessels growing through the device.

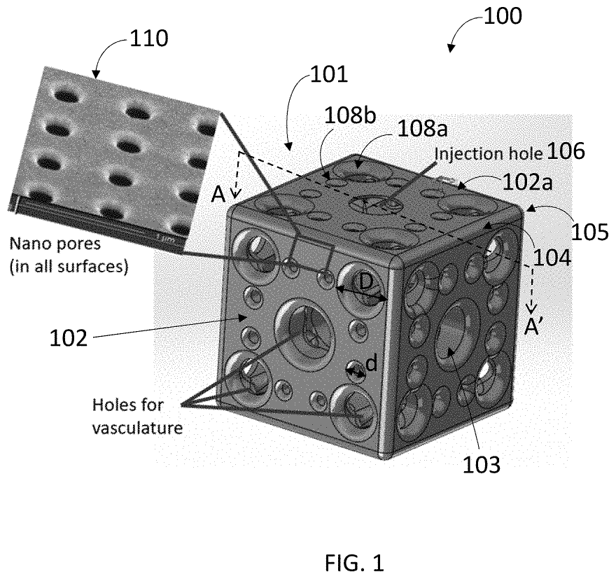

Additional secondary gains can be obtained from application of arbitrary geometry to the design and manufacture of the disclosed encapsulation devices. Given the intricate topography of organs within a body of a subject, shaping and designing the cell encapsulation device to the specific cavity in which it will reside will result in superior outcomes. For example, it has been shown that curvature, size and surface texture are all factors in whether or not a foreign body is rejected. Thus, by taking all these factors into account during the design and manufacture of the device, adverse immune responses to the implanted device can be reduced. As one example, the filleting of edges, corners, and joints of the device, as described in FIG. 1, results in a smoothening of the device's surface and higher biological compatibility.

Example embodiments of an encapsulation device are disclosed wherein the device includes an attachment mechanism for attaching the device to a target an anatomical structure in the area of the subject's cavity where the device is implanted (e.g., an attachment mechanism that can attach or clip the encapsulation device to a nerve or blood vessel in the subject's cavity).

The disclosed methods for manufacturing of a encapsulation device also rely on an additive process that allows for arbitrarily-complex shapes and features down to 100 nm to 1 μm, or less in size. Therefore, the shape of the device including the network of channels traversing a lumen of the device can be adjusted to match the geometry of the local bodily environment while maintaining smooth contours and appropriately spaced holes for vasculature. At least some of the channels may be configured with fractal geometry resulting in a network of interconnected channels of progressively increasing or decreasing size. This configuration increases the surface area of the device to allow encapsulated cells to remain close to the surface of the device for nutrient access and delivery of therapeutic agents. This fractal configuration also increases contact with endothelial cells, improving vascularization. In addition, due to the ease and speed of manufacture, in one embodiment, an encapsulation device can be printed, cured and seeded during surgery after opening and performing a topographical scan of the region where the device is to be implanted. This enables the device to be printed so as to have a shape (e.g., curvature) matching the topography of the region where it is to be implanted.

II. Description of Particular Embodiments

FIG. 1 shows an exemplary embodiment of encapsulation device 100 in accordance with the present disclosure. The device has a closed polygonal shape 101 defined by a plurality of walls 102. Each of the plurality of walls 102 is coupled to an adjacent wall along edge 104, connecting at joint 105. In the depicted embodiment, the polygonal shape is cuboid. However, this is not meant to be limiting. The shape may be a different polygon, such as a tetrahedron, pentahedron, hexahedron, heptahedron, pyramid, dodecahedron, octahedron, etc.

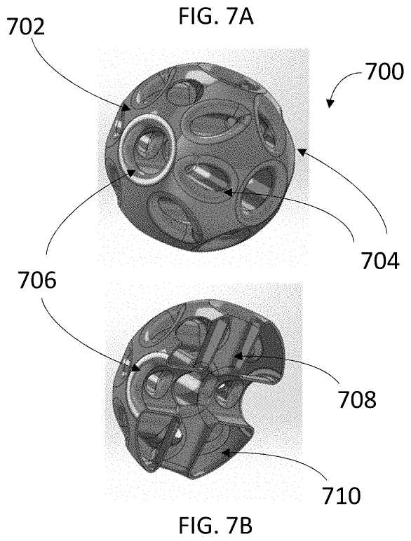

In other embodiments, as described at FIGS. 7A-9, the shape of the device may be a spherical polygon, such as a sphere, an ovoid, a dumbbell, dome, etc. Further still, the shape of the device may be non-uniform or irregular. As such, a shape of the device may be selected based on various factors including one or more of a function of the device (e.g., whether the device will be releasing a drug, a hormone, a growth factor, a peptide, etc., or whether the device will be housing a sensor), location where the device is to be implanted (e.g., in a large blood vessel, under the skull, at an organ such as a kidney or liver, etc.), and nature of the payload that the device is encapsulating (e.g., stem cells, islet cells, bone cells, etc.). As non-limiting examples, a device that will be implanted near, or attached to, a hepatic portal vein, or a device that is to be seeded with insulin-secreting cells (such as islet cells), may be configured with a spherical or cuboid shape. In comparison, a device that will be transplanted at a location between blood vessels may be dumbbell or peanut shaped. As such, any shape may be generated based on the cavity into which the device will be transplanted.

Based on the shape, the device may have a plurality of distinct walls 102, such as in the case of a cuboid shape. Alternatively, the wall 102 may be a continuous surface, such as in the case of a spherical shaped device.

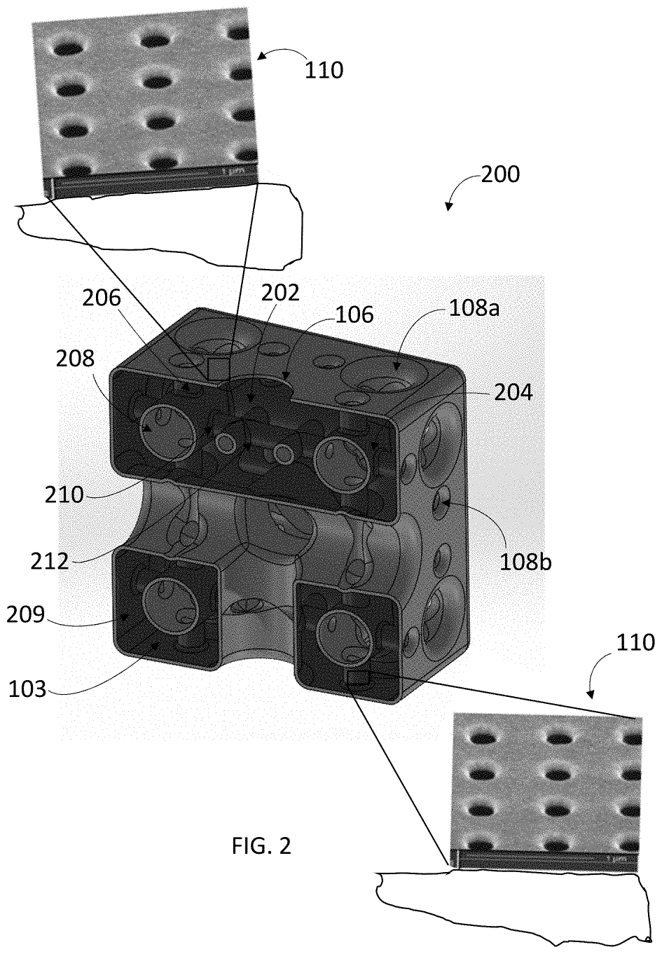

In embodiments having a plurality of distinct walls 102, the walls may be coupled along edges 104 and joints 105. Edges 104 and joints 105 may be filleted to provide a smooth surface, enhancing the device's biological compatibility. In other embodiments, the edges may be chamfered, beveled, or rounded. Together, the plurality of walls 102, edges 104, and joints 105 of the polygonal structure 101 enclose, and thereby define, an inner volume (103, FIG. 2) of the device.

The plurality of walls 102 are about 1 μm to about 20 μm thick, such as about 1 μm to about 5 μm thick, about 2.5 μm to about 10 μm thick, about 5 μm to about 15 μm thick, or about 10 μm to about 20 μm thick (for example, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, or about 20 μm thick). In some examples, the thickness of the walls may be such that, at any given time following implantation, cells seeded into the device are no more than about 40 μm, or about 3 cell bodies, away from a subject's vasculature.

Each of the plurality of walls 102 have a number of vasculature holes 108 that enable fluidic communication through the device from the medium in which the device is implanted. In other words, the vasculature holes 108 couple the inner volume of the encapsulation device with a medium or environment external to the encapsulation device. The vasculature holes 108 may be of varying size including larger vasculature holes 108a having a larger diameter “D” and smaller vasculature holes 108b having a smaller diameter “d.” Holes of one or more intermediate size, with diameters having any value intermediate diameters d and D, may also be present.

In some embodiments, the vasculature holes may have a diameter of about 1 μm to about 1 mm. As an example, vasculature holes may range from about 1 μm to about 50 μm, such as about 3-10 μm, about 5-10 μm, about 10-20 μm, about 15-25 μm, about 20-30 μm, about 25-35 μm, about 30-40 μm, about 35-45 μm, or about 40-50 μm (for example, about 10 μm, about 15 μm, about 20 μm, about 25 μm, about 30 μm, about 35 μm, about 40 μm, about 45 μm, or about 50 μm). In other examples, one or more of the vasculature holes may be at least about 1 μm, at least about 3 μm, at least about 5 μm, at least about 10 μm, at least about 20 μm, at least about 30 μm, at least about 40 μm, at least about 50 μm, or more in diameter. In further examples, one or more of the vasculature holes may be about 5 mm or less, such as about 4 mm or less, 3 mm or less, 2 mm or less, 1 mm or less, 500 μm or less, 300 μm or less, 200 μm or less, 100 μm or less, 50 μm or less, 30 μm or less, 10 μm or less, 5 μm or less, or 3 μm or less in diameter.

The size of the vasculature holes may also be a function of the dimension of the encapsulation device, such as less than a defined percentage of a length of edge 104, such as less than 50%, less than 25%, or less than 10%. Each wall 102 may comprise multiple vasculature holes. In some embodiments, all vasculature holes of a device may be of a uniform size. In other embodiments, each wall may comprise holes of differing size, such as multiple larger holes 108a interspersed with multiple smaller holes 108b. In still other embodiments, a given wall may only have holes of a defined size. For example, some walls of the device may only have larger vasculature holes 108a while other walls may only have smaller vasculature holes 108b.

The vasculature holes 108 may have fractal or self-similar geometry. Thus, vasculature holes of a continuum of length scales can be provided, down to a cellular scale. As an example, an encapsulation device that is a cubic centimeter in size may have vascular holes of 6-10 length scales, while another encapsulation device that is a cubic millimeter is size may have vascular holes with 3-5 lengths scales. It will be appreciated that the device of FIG. 2 discloses just one example at a given scale, showing vascular holes of about 3 length scales. However, different length scales (including combinations of length scales) may be possible without departing from the scope of the disclosure.

The device may also include vasculature holes 108 of varying sizes of a continuum of sizes or length scales. As an example, on a centimeter scale device, the largest vasculature hole may be 3000 microns, followed by vasculature holes of a continuum of length scales such as 1000 microns, 300 microns, 100 microns, and 30 microns. As another example, a device sized to a cubic millimeter may have vascular holes of size 300 microns, 100 microns, and 30 microns. Thus, in some example embodiments of an encapsulation device, a regular sequence of vasculature hole sizes can be provided that is based on a starting length scale of the device. The general form of this fractal geometry provides various advantages to the device without being limited by the exact sizes or exact geometric sequence of the hole sizes. In further examples, a vascular hole may be as large as an entire wall surface.

The vasculature holes 108 may be provided in any arrangement on the wall 102. For example, the vasculature holes may be arranged symmetrically to provide a desired pattern. Alternatively, the vasculature holes may be arranged asymmetrically, for example, randomly dispersed across wall 102. In the configuration depicted for the example device of FIG. 1, each wall of the cuboid device 100 has four larger holes 108a arranged at each of 4 corners of the surface (adjacent to corresponding joints 105) and a central larger hole (also referred to herein as a central vasculature hole) arranged at a center of the wall. Further, a total of 8 smaller holes 108b are arranged symmetrically around the central larger hole. In other embodiments, there may be a smaller or larger number of vasculature holes. Vasculature holes on a first wall of the device may be arranged in the same position as vasculature holes on a second wall, opposing the first wall. In other embodiments, vasculature holes on opposing walls may be offset relative to one another. As elaborated with reference to FIG. 2, a symmetric arrangement of vasculature holes allows for the use of fractal geometry in creating a cage structure inside the inner volume of the device. The cage structure increases the surface area of the device where vascularization of seeded cells can occur.

The encapsulation device 100 may also have an injection port 106 for receiving a payload. Typically, the device may have only a single injection port. For example, a top wall 102a of the device may include the injection port 106. In other embodiments, there may be a larger number of injection ports (for example, two injection ports in a dumbbell shaped device, etc.). However, a total number of injection ports may be smaller than a number of vasculature holes of the device. The injection port 106 may have a shape that can be circular, square, rectangular, or any other regular or irregular shape. In some examples, such as where a single injection port is provided on a top wall, the injection port may be positioned at a central location (for example, a center of the top wall, or a center of the arrangement of vasculature holes on the top wall). The injection port 106 may be configured to have a diameter (or width, if the port is not circular) that is the same as, smaller than, or larger than a diameter of the larger vasculature holes 108a. The upper limit of the injection port size may include the full top surface of the encapsulation device. In such an embodiment, the full top surface of the device may be the port for receiving a payload. In one example, the injection port has a diameter of about 100 μm. A payload (e.g., biological cells or a hydrogel embedded drug) may be seeded into the inner volume of the device 100 via the injection port 106.

Each wall 102 of the device is fabricated to have a continuously porous structure to allow small molecules to pass freely across the wall. The porous structure may include micropores or nanopores. In the depicted example, the wall 102 has nanopores 110 which allows for small molecules such as hormones (e.g., insulin), drugs, and nutrients (e.g., glucose) to freely pass between a medium external to the device and the inner volume of the device across the wall 102. The size of the nanopores may restrict infiltration of larger molecules, such as antibodies and other immune response factors. As a result, contents of the encapsulation device 100 are protected from factors that could trigger an adverse immune response to the implanted device. In one example, the size of the pores is less than about 2 μm (such as less than about 1.5 μm, less than about 1 μm, less than about 0.5 μm, less than about 0.1 μm, or less than about 0.05 μm. In some examples, the size of the pores may be in the range of about 0.02 μm to about 2 μm in diameter (for example, about 0.02 μm to about 0.1 μm, about 0.05 μm to about 0.2 μm, about 0.1 μm to about 0.5 μm, about 0.25 μm to about 1 μm, about 0.75 μm to about 1.5 μm, or about μm to about 2 μm). This pore size allows the contents of the device (such as cells seeded into the device) to remain contained in the device, while being protected from detection by the immune system. In some embodiments, the pores of the continuously porous structure have a uniform pore size. In other example embodiments, the pores of the continuously porous structure may have varying pore size.

The encapsulation device 100 may be fabricated using 3D printing using, for example, multi-photon stereolithographic techniques. The fabrication method uniquely allows for the creation of multi-scale channels on the interior of the device. In particular, the 3D printing method facilitates the creation of the fractal structure internal to the encapsulation device. Also, as a result of the fabrication method, features less than 0.1 μm in size, or in the range of the nanopores, can be generated. Further, 3D printing allows the shape of the device 100 to be customized to any desired shape (e.g., regular or irregular shape) and size. In some examples, the encapsulation device is fabricated to be between 0.0003 and 10 cm³ in size (for example, about 0.001 to about 0.01 cm³, about 0.01 to about 0.1 cm³, about 0.1 to 1 cm³, about 1 to 2.5 cm³, about 2 to 5 cm³, or about 5 to 10 cm³ in size). FIG. 2 shows a cross-sectional view 200 of the encapsulation device 100 of FIG. 1 along axis A-A′. Each vasculature hole 108a, 108b on the wall 102 of the device extends into a corresponding channel. A diameter of the channel matches the diameter of the vasculature hole it extends from. Thus, larger vasculature holes 108a extend into wider channels 204 while smaller vasculature holes 108b extend into narrower channels 206, and so on.

In one embodiment, the channels 204, 206 run straight though the encapsulation device. For example, the channels may run parallel to a longitudinal and/or a transverse axis of the device. The channels 204, 206 may run along straight lines through the inner volume 103 of the device. For example, wider channels 204 may extend from a larger vasculature hole 108a on a first wall, extending through the inner volume 103, and connecting to another larger vasculature hole 108a positioned on a second wall, opposite the first wall. Likewise, narrower channels 206 extend from a narrower vasculature hole 108b on a first wall, extending through the inner volume 103, and connecting to another smaller vasculature hole 108b positioned on a second wall, opposite the first wall. In this way, channels couple vasculature holes on opposing walls with each other. In other embodiments, channels 204, 206 may extend from a first wall to a second wall that is not opposite to the first wall (e.g., the channel may turn within the device). For example, channels 204, 206 may extend from a first wall to a second wall (for example, an adjacent wall or an orthogonal wall).

In some examples, as shown, channels may couple a first vasculature hole to another vasculature hole. In other embodiments, at least a portion of the channel may be branched, thereby coupling a plurality of vasculature holes (e.g., holes of one wall) to a single vasculature hole (e.g., a hole of an adjacent, opposing, or orthogonal wall). For example, the channels may form a T-shaped or Y-shaped connection, thereby connecting the vasculature holes internally through the device. In some embodiments, such as where the channels are serpentine or curved, the channels may couple a vasculature hole of a given wall with another vasculature hole of a different or the same wall. For example, a channel extending from a vasculature hole of the wall may have a path through an inner volume of the device and couple to another vasculature hole of the same wall, such as a vasculature hole positioned adjacent to the hole the channel originally extended from.

By enabling vasculature holes of different sizes, and at different positions on the surface of the device, to be coupled internally via the channels, the various channel configurations can be used to create specific diffusion paths. In one example, as discussed below, multiple encapsulation devices, each seeded with distinct payloads and each releasing distinct molecules, can be assembled or aligned together such that a target flow path can be provided through the devices.

As a result of the channels, a medium external to the encapsulation device can enter the device through a vasculature hole (such as a vasculature hole on a wall), flow through the device via the channels, and then flow out of the device through another vasculature hole (such as a vasculature hole on another wall, e.g., an opposing wall). In other examples, cells (such as vascular endothelial cells) can enter the device through a vasculature hole and can produce vascularization of one or more channels.

In another embodiment, the channels 204, 206 may be interconnected to create a lattice or cage-like structure inside the inner volume of the encapsulation device.

In still additional embodiments, the channels 204, 206 may run straight and additionally interconnect. For example, in some embodiments, channels extending from vasculature holes on orthogonal walls (that is, walls that are at right angles to each other in the depicted cuboid device) or adjacent walls may also be interconnected within the inner volume 103. Thus, wider channels 204 may be in fluidic communication with other wider channels as well as with narrower channels 206 within inner volume 103 to create a fractal structure 202. For example, a wider channel 204 may be coupled to a narrower channel 206 at a first junction 210, and a narrower channel 206 may be coupled to another narrower channel 206 at a second junction 212. Multiple channels may intersect at a given junction, generating a lattice-like connection of channels. In one example design embodiment, channels of length scale n intersect on the interior of the device with larger channels of length scale n+1.

While the channels are depicted in FIG. 2 as running straight, it will be appreciated that in alternate embodiments, the channels may be arched, curved, or serpentine. For example, a channel extending from a vasculature hole may take a curved path through an inner volume of the device before being coupled to another vasculature hole on the same or a different outer wall of the device.

In further embodiments, the channels may have a shape that follows a contour of the walls. A length of channels 204, 206 may be in the range of about 10 to 50 μm, for example. Further, the length of the channels may be a function of the overall dimensions of the device. In one example, the channel sizes may scale in a geometric progression as n/3, n/(3²), n/(3³) . . . down to 3-5 microns, starting with n, where n is the size of an edge of the device. In another example, the channel sizes may scale in a geometric progression as n/4, n/(4²), n/(4³) and so on, or n/5, n/(5²), n/(5³) and so on, down to 3-5 microns, starting with n, where n is the size of an edge of the device. In this way, the channel sizes may scale in a geometric progression as n/m, n/(m²), n/(m³) . . . down to 3-5 microns, starting with n, where n is the size of an edge of the device and m is an integer.

A diameter of channels 204, 206 may be in the range of about 10 μm to about 50 μm in diameter, such as about 10-20 μm, about 15-25 μm, about 20-30 μm, about 25-35 μm, about 30-40 μm, about 35-45 μm, or about 40-50 μm (for example, about 10 μm, about 15 μm, about 20 μm, about 25 μm, about 30 μm, about 35 μm, about 40 μm, about 45 μm, or about 50 μm).

Each of the channels is also fabricated using 3D printing. By relying on 3D printing, and techniques such as multi-photon stereolithography, each of the channels can be fabricated to include the same porous structure 110 (e.g., microporous or nanoporous structure) as walls 102. The porous nature of the channels allows nutrients (e.g., from the bloodstream) to feed cells that are enclosed in the device, and/or for molecules originating from within the device to diffuse out into the pore channels.

Alternatively, distinct walls 102, and/or distinct sets of channels may be fabricated to include distinct porous structures. For example, a pore size on a top surface of the device may differ from the pore size of a side surface or bottom surface of the device. A thickness of the channel walls may be the same as, or different from, the walls 102. As an example, walls 102 and channels may have a uniform thickness of between 1 to 20 μm. In other examples, the thickness of walls 102 may be greater or less than the thickness of channel walls. Further still, the walls of a first set of channels may have a thickness that is different from the walls of a second, different set of channels.

As a result of the cage structure 202 generated via the channels (e.g., intersecting channels 206, 208), the inner volume 103 of the device is divided into an outer lumen 208 (lighter shading) and an inner lumen 209 (darker shading). The outer lumen 208 defines a region of the inner volume that is enclosed within all the channels 204, 206 and is in fluid communication with the outer environment. The inner lumen 209 defines a region of the inner volume that is enclosed within walls 102 but outside of channels 204, 206. Outer lumen 208 is in fluidic communication with a medium in which the device is placed, via vasculature holes 108a, 108b. For example, cells in the medium (e.g., endothelial cells), which are in contact with the wall 102 of the encapsulation device 100, can enter the outer lumen 208 through vasculature holes 108a and/or 108b. The cells can then vascularize through the channels 204, 206. At the same time, the cells entering the channels are prevented from entering inner lumen 209.

Since the channels 204, 206 also contain pores, nutrients (such as oxygen, glucose, etc.) can pass from the medium or vascular cells in the channels into the inner lumen 209 and molecules (e.g., a protein, such as insulin) can pass from the inner lumen 209 into the channels. The structure thereby provides more nutrient and therapeutic molecule transport per cell volume. Consequently, a greater number of cells can be seeded in the encapsulation device without necrotic die-off, and more therapeutic molecules per cell can reach the blood stream of a subject into whom the device is implanted.

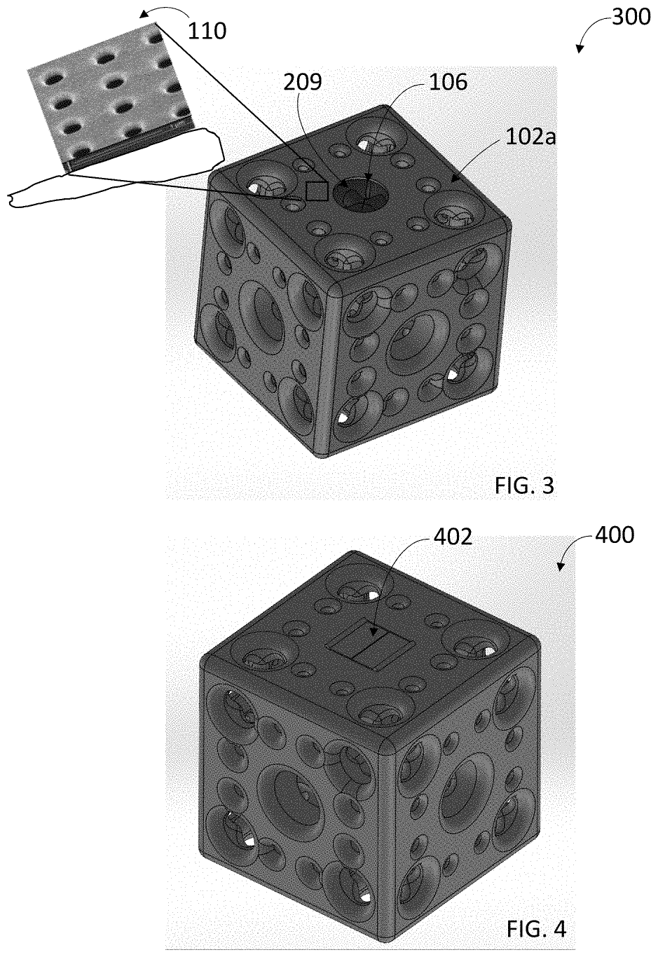

Inner lumen 209 is in fluidic communication with the medium in which the device is placed only via injection port 106. Thus, a payload (e.g., cells, a drug, a therapeutic substance, or other biological sample) can be seeded into the device 100 via injection port 106. Therein, the payload remains in the inner lumen 209, as shown in FIG. 3. The injection port can be sealed, for example using a trap door 402 as shown in FIG. 4, or other closure mechanism (such as a flap), encapsulating and retaining the payload in the inner lumen 209.

A relative size of channels 204, 206 and vasculature holes 108a, 108b may be based on fractal geometry. In particular, the channels may include a repeating pattern that displays at every scale. This includes larger channels connecting into a network of channels of progressively decreasing size, and smaller channels connecting into a network of channels of progressively increasing size. The expanding symmetry provided by the fractal geometry results in a highest surface area of contact for the channels while also maintaining a highest volume, irrespective of an external shape of the encapsulation device. In other words, the volume of outer lumen 208, which is the space where vascularization occurs, is increased. As a result of the configuration of the interconnected channels, as well as the thickness of the walls and the porous nature of the outer walls and channels walls, at any given time, cells seeded inside the encapsulation device, after the device is implanted into a cavity of a subject, are about 3 cell bodies (˜40 μm) away from the subject's vasculature. By providing a cage structure of channels having fractal geometry, the vascularization efficiency of cells within outer lumen 208 is improved. In addition, the surface area of the outer lumen 208 (that is, the surface of the cage structure defined by the inner portion of the channels) is increased, enabling higher diffusion rates across the channels.

FIG. 3 shows a top perspective view 300 of an encapsulation device. As revealed in this view, injection port 106 in the center of top wall 102a is fluidically coupled only to inner lumen 209. A payload seeded through injection port 106 remains outside of the channels 204, 206, but within the walls of the encapsulation device. The walls of the device have a porous structure 110 (as exemplified by top wall 102a), in addition to the presence of vasculature holes.

Due to the additional porous nature of the channels (e.g., as shown in FIG. 2), small molecules can cross the channel surface allowing for selective fluidic communication between the contents of the outer lumen 208 and the contents of the inner lumen 209. For example, small molecules secreted by the payload seeded into the device can pass from the inner lumen 209 into the network of channels 204, 206, and thereby into outer lumen 208. Likewise, small molecules secreted by cells vascularizing through the network of channels can pass from the outer lumen 208 into the inner lumen 209. Further, small molecules such as nutrients and drugs can pass between the medium in which the device is implanted and the inner lumen 209 and outer lumen 208 of the device. At the same time, the payload seeded into inner lumen 209 cannot enter the network of channels 204, 206, or outer lumen 208, or be discharged outside of the device 100. Likewise, cells vascularizing through the network of channels 204, 206 cannot enter the inner lumen 209. Furthermore, larger immune molecules or cells, such as antibodies and leukocytes, cannot enter the inner volume of the device, reducing the likelihood of an adverse immune response to the implanted device.

Injection port 106 may include a closure mechanism in order to be sealed to isolate the contents of inner lumen 209 from a medium outside the device. The injection port 106 may be designed to have a shape that can be circular, square, rectangular, or any other regular or irregular shape. In some examples, the shape of the injection port may be a function of an overall shape of the encapsulation device, such as a circular injection port for a spherical device, a square or rectangular injection port for a polygonal device, etc. A position of the injection port 106 on a wall 102 of the encapsulation device may also be a function of an overall shape and design of the encapsulation device. For example, in non-spherical polygonal embodiments (such as the cuboid device of FIG. 1), the injection port may be configured on a top wall. Further, in embodiments where a single injection port is provided, the injection port may be positioned at a central location of the top wall, such as at a center of the arrangement of vasculature holes on the top wall. It will be appreciated that in still further examples, the encapsulation device may be designed with one or more of a position, size, and shape of the injection port selected as a function of the payload (e.g., based on whether the payload includes biological cells or a hydrogel-embedded drug).

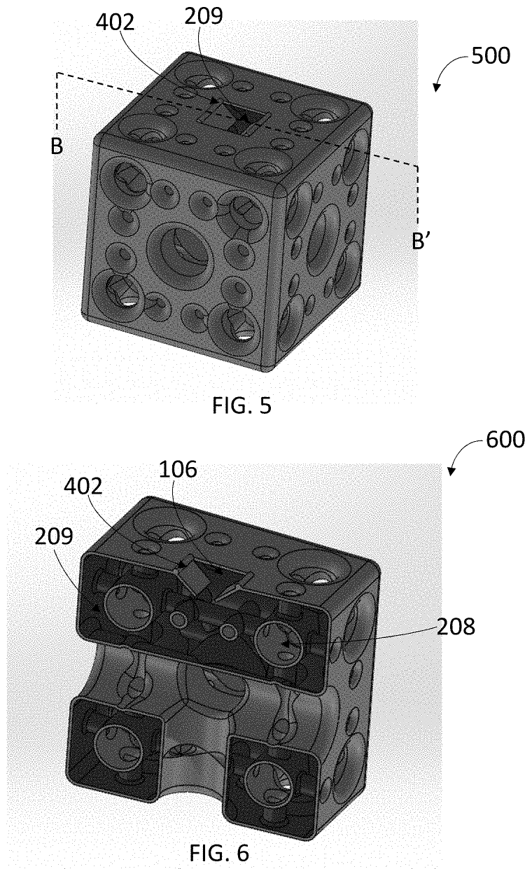

Various closure or sealing options are envisioned. As a non-limiting example, depicted at FIG. 4, a trap door 402 may be printed into or onto injection port 106. In one embodiment, the trap door may flip downwards (view 500, FIG. 5) during a seeding procedure. Then, after injection of the payload, the trap door may flip upwards (FIG. 4), adhering to the wall it is mounted on. In other examples, the trap door may flip upwards during seeding and then close by flipping downwards. Edges of the trap door may be sealed with a sealant, such as an epoxy, photocured glue, or other known methods. Still other sealing options may be possible such as non-biodegradable hydrogel. For example, a trap door can be sealed by laser welding a cap over the injection port. In a non-limiting example, one potential polymer substrate for the device is the hybrid material Ormocomp which forms a silicon oxide (glass) network upon polymerization. A thin glass coverslip may be placed over the injection port and the two surfaces welded together using an ultrafast laser.

FIG. 6 shows a cross-sectional view 600 of the device of FIG. 5 through the trap door, along axis B-B′. A payload is seeded into the device, and particularly into inner lumen 209 of the device, via injection port 106 when trap door 402 is opened. If biological cells are the payload, the encapsulation device may be placed in cell culture media and dissociated cells can be injected through the injection port using a microinjector and/or a small pipette after which the injection port is sealed by closing the trap door. After seeding, the cells may be incubated in vitro by immersing the seeded encapsulation device in a cell culture medium prior to implantation. The seeding process may be performed manually, or automated using available seeding technology.

If drugs are the payload, the drug may be injected directly or in the form of a time-release medium, such as hydrogel, from which it can diffuse out, for example to a bloodstream or to a cell culture medium.

Once the payload has been seeded, the entire encapsulation device can be implanted into a subject at a desired location. Due to the small pore size of the encapsulation device, once seeded, the contents will not leak out of the inner volume 103 of the device. As a result, if desired, the entire encapsulation device with its contents can also be easily explanted. In some cases, the device may be printed with a fluorescent material (such as fluorescein) in the printing media to assist in locating the device (e.g., for explanting, if necessary).

In some embodiments, all surfaces of the encapsulation device, including surfaces of the walls 102 and channels 204, 206, may be coated with factors that improve cellular adherence. For example, prior to seeding and implantation, all surfaces of the encapsulation device may be coated with cellular adherence proteins or extracellular proteins such as polylysine, collagen, laminin or fibronectin to promote adhesion during seeding. Generally, extracellular proteins or glycoproteins can be used for coating the device to favor cellular adhesion to the surface of the device. In further embodiments, antibodies can also be coated on the surface of the encapsulation device to capture specific cell surface marker-expressing cells onto the device. The surface coating may also be selected to limit foreign body response to the device following implantation. In some examples, the channels are non-covalently coated with growth factors, such as vascular endothelial growth factors, to promote endothelial growth of cells through the channels. In some embodiments, growth factors may be included as an additional agent with the payload. Therein, the growth factors may be seeded with the cells into the lumen of the device via the injection port. From the seeding location, the growth factors may diffuse outward to attract endothelial cells to the surface of, or into, the device.

FIG. 7A shows another example embodiment 700 of an encapsulation device having a spherical structure. The device has a continuous outer spherical wall 702 enclosing an inner volume of the device. The outer spherical wall comprises a single injection port 706 surrounded by a plurality of vasculature holes 704 of varying diameter. The vasculature holes 704 may be distributed evenly throughout the outer spherical wall 702. The spherical structure also includes micro- or nano-pores (not shown).

FIG. 7B shows a cross-sectional view of the embodiment of FIG. 7A. The vasculature holes 704 extend into a plurality of channels 708 traversing a lumen 710 of the device. The channels 708 run diametrically through the inner volume of the spherical structure, extending from a given vasculature hole to a diametrically opposite vasculature hole. The single injection port 706 is not coupled to the volume defined by the channels but is coupled to the lumen 710 of the device, external to the interconnected channels 708, but internal to the outer spherical wall of the device.

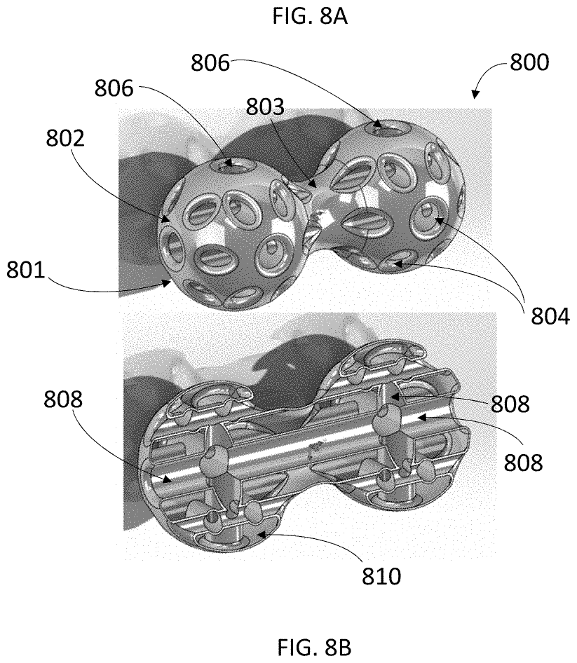

FIG. 8A shows yet another example embodiment 800 of an encapsulation device having a dumbbell structure. The dumbbell structure is defined by a continuous outer wall 801 including two spherical wall sections 802 separated by a cylindrical connecting wall section 803. Each of the spherical wall sections 802 comprises a single injection port 806 for receiving a payload. Further, each of the injection ports 806 are surrounded by a plurality of vasculature holes 804 of varying diameter. At least some of the vasculature holes 804 extend from the spherical wall section 802 into the cylindrical connecting section 803. The dumbbell structure also includes micro- or nano-pores (not shown).

FIG. 8B shows a cross-sectional view of the embodiment of FIG. 8A. The vasculature holes extend into a plurality of channels 808 traversing a lumen of the device. At least some of the channels 808 run diametrically through the inner volume of a corresponding spherical wall section, extending from a given vasculature hole of the spherical wall section to a diametrically opposite vasculature hole of the same spherical wall section. At least some of the channels 808 run along a length of the dumbbell structure, extending from a given vasculature hole of a first spherical wall section to a diametrically opposite vasculature hole on a second spherical wall section, while also extending through a length of the intermediate cylindrical connecting section 803. The single injection ports 806 are not coupled to an inner volume defined by the interconnected channels but are coupled to a lumen 810 of the device, external to the interconnected channels 808, but internal to the outer wall of the device.

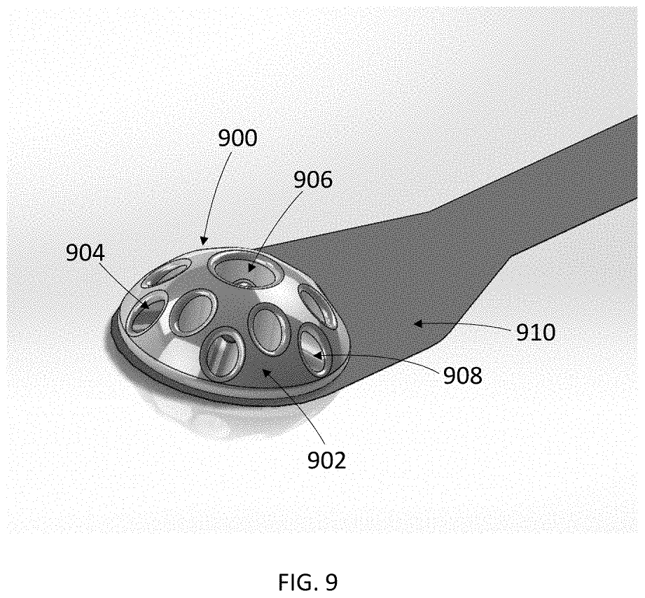

FIG. 9 shows an embodiment 900 of a dome shaped encapsulation device having a dome-shaped wall 902 extending into a spherical base wall (not shown in this view) on an underside of the device, the wall encapsulating an inner volume of the device. A plurality of vasculature holes 904 extend from wall 902 into a plurality of channels 908 traversing the inner volume of the device. At least some of the channels 908 run diametrically through the inner volume of the device, extending from a vasculature hole of the dome shaped wall 902 to a diametrically opposite vasculature hole of the wall 902. Injection port 906 is provided on a top (or crown) of the dome and is selectively coupled to an inner lumen of the device, external to the channels 908, but internal to the wall 902 of the device. The structure also includes micro- or nano-pores (not shown). In the depicted embodiment, the encapsulation device is manufactured on a tether 910 that is left attached to the device for ease of implantation.

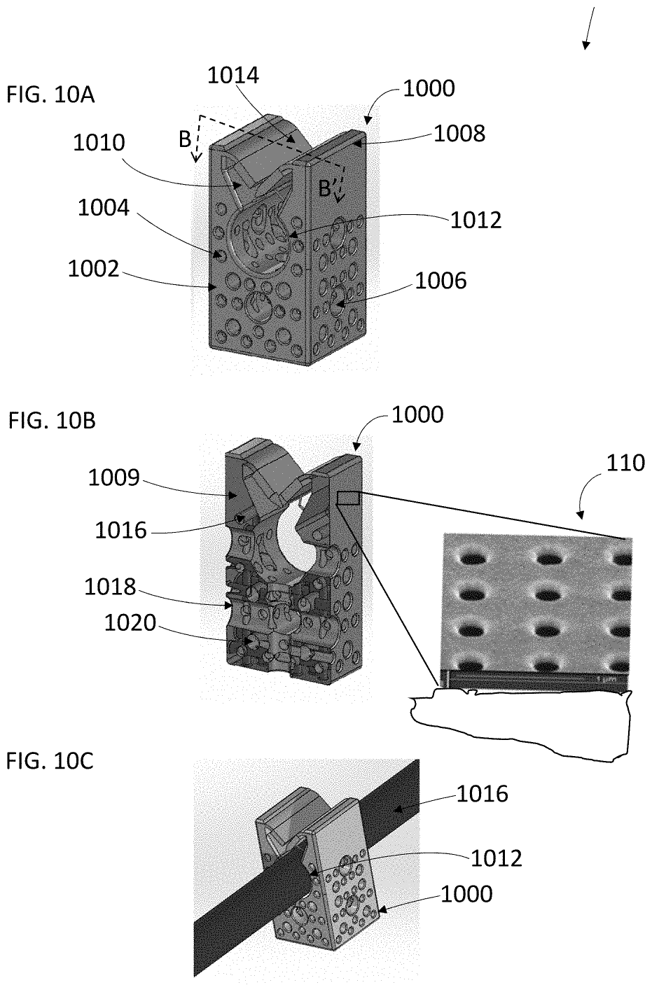

FIGS. 10A-C depict an example embodiment 1000 of a substantially quadrangular encapsulation device further comprising an attachment mechanism for attaching the device to a target anatomical structure (e.g., an attachment mechanism that can clip the encapsulation device onto a nerve or blood vessel in the subject's cavity).

FIG. 10A shows a perspective view of encapsulation device 1000 having outer walls 1002 configured with a plurality of vasculature holes 1004, 1006 of varying sizes. The device further includes an injection port for receiving a payload. In the depicted embodiment, the injection port is on a bottom wall of the device and is therefore not shown. The device 1000 includes an attachment mechanism 1014 for coupling the device to a target anatomical structure in a subject. For example, the attachment mechanism allows the device to be attached to a blood vessel or nerve, thereby increasing the efficiency of a localized treatment provided by the payload seeded into the device. Attachment mechanism 1014 comprises a recess 1012 and a seal 1014.

Recess 1012 extends from a top wall 1008 of the device towards a bottom wall (e.g., the wall with the injection port) and defines a volume within which the specific anatomic structure is received. A shape and size of the recess is configured to accommodate the specific structure the device is to be attached to. In the depicted example, the recess is substantially spherical in shape and extends a distance that is about halfway between the top wall and the bottom wall. Particularly, the top wall 1008 has a filleted edge which then extends into a tapered wall, the tapered wall then extending into an inner surface of a spherical cavity. In other embodiments, the recess may have a different shape, a different angle of the tapered wall, may extend a different distance between walls, and/or may define a different volume.

Seal 1014 extends from the top wall 1008 towards the recess, along the tapered wall. Seal 1014 can be reversibly opened and closed. In the depicted embodiment, the seal is configured as a trap door which opens downwards (towards the recess) to provide access to the spherical cavity, and closes upwards (away from the recess). In other examples, the trap door may open upwards and close downwards, or may be configured as a clip.

After a payload has been injected into the device 1000, seal 1014 may be actuated to an open setting to position the recess of the device around the anatomical structure 1016. FIG. 10C shows device 1000 with a blood vessel accommodated into recess 1012. Then, the seal may be actuated to a closed setting, leaving the device attached to the biological structure 1016.

FIG. 10B shows a cross-sectional view of the embodiment of FIG. 10A along axis B-B′. The vasculature holes 1004, 1006 extend into a plurality of channels 1016, 1018 traversing an inner volume of device 1000 and separating an inner lumen 1009 (darker shading) from an outer lumen 1020 (lighter shading). The inner lumen defines a volume internal to the device but external to the channels 1016, 1018, and therefore separated from a medium outside the device by nanoporous wall 1002. The outer lumen defines a volume internal to the channels, and fluidly connected to a medium outside the device.

Attachment of the device 1000 to the anatomic structure 1016 (in this example, a blood vessel) results in transfer of material, across the porous structure 110 of the wall 1002, between blood vessel 1016 and the inner lumen 1009. The proximity to the biological structure can enhance vascularization efficiency through the device and overall local therapeutic efficacy of the payload used to seed the device.

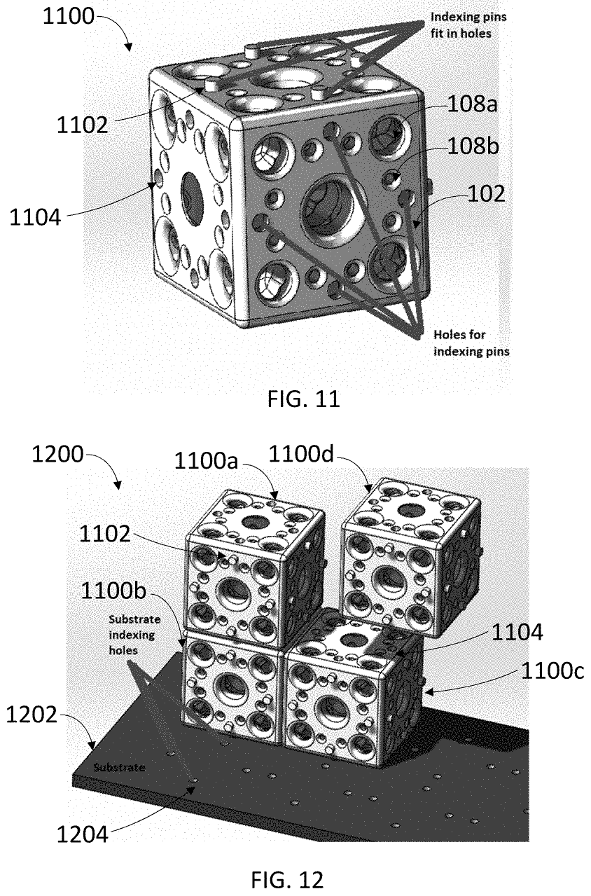

In further embodiments, as described with reference to FIGS. 11-12, one or more non-vascular structures can be printed inside or outside of the encapsulation device. These non-vascular structures may include, as non-limiting examples, solid columns, beams, struts, wall thickness changes, scaffolds, protuberances, pins, sockets, or holes. The non-vascular structures may be included to provide structural stability to the device. Further, the non-vascular structures may enable coupling of one device to another, or to a substrate.

In some embodiments, the non-vascular structures may be provided on an outer surface of the walls of the device. These non-vascular structures are thus exposed to an external medium. In other embodiments, the non-vascular structures may be additionally or alternatively provided on an inner surface of the walls of the device, and thereby exposed to cells seeded inside the inner lumen of the device. In still further embodiments, the non-vascular structures may be additionally or alternatively provided on the outer surface of the channels, and thereby exposed to cells seeded inside the inner lumen of the device. In yet another embodiment, the non-vascular structures may be additionally or alternatively provided on the inner surface of the channels, and thereby exposed to cells and materials entering the device from the external medium via the vasculature holes.

The non-vascular structures may be arranged symmetrically or asymmetrically relative to device features, such as relative to vasculature holes, or channels. For example, the non-vascular structures can be positioned to align vasculature holes of a first device with vasculature holes of a second device to promote vascularized connections between the first device and the second device. In other examples, the non-vascular structures may be printed on a surface of an outer wall of an encapsulation device and positioned so as to align vasculature holes of the device with a substrate.

In some examples, a given device may include only a single type of non-vascular structure (e.g., only sockets or only pins). In other examples, a given device may include multiple types of non-vascular structures (e.g., both sockets and pins). The multiple types of non-vascular structures may be distributed evenly or unevenly across device features or distinct non-vascular structures may be provided on distinct device features. For example, walls of the device may include a different non-vascular structure than the lumen of the device (e.g., scaffolds in the lumen and pins on the walls).

In some examples, each wall of the device may comprise each of the multiple types of non-vascular structures arranged symmetrically or asymmetrically thereon. In other examples, some walls of the device may include a first type of non-vascular structure (e.g., only sockets) while other walls include a different type of non-vascular structure (e.g., only pins).

In some examples, a first set of non-vascular structures may be provided on a first device (such as a first set of non-vascular structures printed on a surface of an outer wall of a first device) and a second set of non-vascular structures may be provided on a second device (such as a second set of non-vascular structures printed on a surface of an outer wall of a second device), the first set of non-vascular structures configured to connect with (e.g., form a mated connection with) the second set of non-vascular structures, thereby coupling the first device to the second device to promote vascularized connections between the first device and the second device. The arrangement of non-vascular structures can allow for easy alignment and juxtaposition of a first device with another device, such as by engaging pins on the wall of a first device with the sockets on the wall of a second device.

A combination of the arrangement of channels provided within each encapsulation device with the arrangement non-vascular structures can be leveraged to create specific diffusion or flow paths for nutrients and biological molecules through the device. For example, by configuring the device with channels that internally couple vasculature holes of different sizes, and at different positions on the surface of the device, various channel configurations can be provided. By further assembling multiple encapsulation devices, and positioning them to connect vasculature holes and/or channel configurations of one device with those of another device, a target diffusion path can be created starting from one device and flowing through another device. Distinct devices may be seeded with distinct payloads and each may be releasing distinct molecules, resulting in synergistic benefits. For example, devices “A” and “B” producing molecules “a” and “b” may be assembled to enable the molecules to be delivered jointly to a tissue. In this scenario, channels of device “A” may be aligned or juxtaposed with those of device “B,” allowing the channels to merge prior to delivery of the molecules.

FIG. 11 shows an embodiment 1100 (particularly the device of FIG. 1) with non-vascular structures 1102, 1104 provided on walls 102 of the device. In the depicted example, some walls 102 of the device 1100 include a first set of non-vascular structures 1102 depicted herein as indexing pins or protuberances. Other walls 102 of the device 1100 include a second set of non-vascular structures 1104, depicted herein as indexing holes (or sockets). The first set of non-vascular structures 1102 are configured to be releasably engaged to the second set of non-vascular structures 1104, such as via formation of a mated connection. When engaged, vasculature holes 108a, 108b of a first device may be aligned with (or offset from) vasculature holes 108a, 108b of one or more other devices, or aligned with (or offset from) a substrate feature.

FIG. 12 shows an embodiment 1200 including multiple encapsulation devices 1100a-d having non-vascular structures 1102, 1104. Engagement of non-vascular structures 1102 on a first device 1100a with non-vascular structures 1104 on a second device 1100b results in alignment and juxtaposition of vasculature holes of the first device with those of the second device. Likewise, engagement of non-vascular structures 1102 on the second device 1100b with non-vascular structures 1104 on a third device 1100c results in alignment and juxtaposition of vasculature holes of the second device with those of the third device. As a result of this configuration, even if only the first device 1100a is seeded with a payload, vascularization can extend from the first device to the second device and thereon to the third device.

The encapsulation devices may also be arranged in an offset configuration via engagement of the non-vascular structures. For example, engagement of non-vascular structures 1102 on third device 1100c with non-vascular structures 1104 on a fourth device 1100d results in vasculature holes of the third device being positioned offset from those of the fourth device.

The non-vascular structures can also be used to fixedly position a device relative to a substrate. For example, engagement of non-vascular structures 1102 on devices 1100b and 1100c with non-vascular structures 1204 on substrate 1202 results in the device(s) being held at a target location on the substrate and reduces lateral motion of the device relative to the substrate.

II. Methods of Manufacture

Disclosed herein are methods of manufacturing an encapsulation device of any of the embodiments described above.

The encapsulation devices disclosed herein may be designed with computer-aided drafting software. Various factors may be taken into consideration during the design of the device. For example, a design may be optimized based on one or more of an intended payload, intended implantation location in subject, degree of vascularization desired, intended duration of implantation, etc.

Following design, data pertaining to the design may be saved and converted to a binary file-type within the software, for example to a “.STL” file. The binary file comprising the device design data is then loaded into a separate software package (for example, PrintImage, freely available) that converts the data into binary voxels that identify and encode which spaces within the build volume are to be polymerized. The binary voxel data can then be used for manufacturing the selected design using any known manufacturing methods.

For example, the data may be used to fabricate and polymerize the encapsulation device using additive manufacturing, for example, multi-photon stereolithography. Therein, in brief, an optical objective is dipped into liquid photoresin that fills a build volume. In an example embodiment, the liquid photoresin consists of a polymer (e.g., methacrylated alginate, poly-(ethylene glycol) diacrylate, 2-(Hydroxymethyl)-2-[[(1-oxoallyl)oxy]methyl]-1,3-propanediyl-diacrylate (commercially available), Ormocomp® (available from e.g., Micro Resist Technology, Berlin, Germany), or SU-8 (available from e.g., Micro Resist Technology, Berlin, Germany), cellulose, collagen, chitosan, gelatin methacrylate, or SZ2080, and optionally a photoinitiator such as Irgacure®. In some embodiments, the polymer is doped with small micro or nano-scale solid particles such as silica, carbon nanotubes, or other ceramics, or metals. In some embodiments, a photosensitizer may be additionally used. In some embodiments, a fluorescent material may be added to the resin to enable rapid localization of the encapsulation device after implantation. Still other materials may be added to the resin to assist in localization or identification.

A femto-second-pulsed laser beam (e.g., 200 to 1200 nm beam) is then directed through the objective and rastered through the build volume using a series of high-speed mirrors and lenses. The beam drives polymerization only at the focal point due to the multi-photon effect. The halved wavelength and doubled energy per photon creates the necessary conditions to break secondary chemical bonds thus initiating free radical photopolymerization. A degree of voxel polymerization may be controlled by laser power. Binary switching in Voxelization can be produced by adjusting the setting of the laser relative to (e.g., above or below) a polymerization threshold. This laser power is controlled by a high-speed laser modulator such as an acousto-optic modulator. In addition, the laser modulator adjusts the beam power between the middle and edges of the objective field of view to ensure even polymerization.

To achieve larger encapsulation devices, such as larger than 0.3 mm³ in volume, the stage that the build volume rests on during the fabrication process can be moved or rotated in 6 axes (e.g., X, Y, or Z axes, pitch, roll, and yaw axes). The encapsulation device is then built by polymerizing material at the bottom of the build volume where it rests on a substrate, such as glass, silicone or polyimide. An example of fabrication of the caged structure of the device on polyimide is shown at FIG. 9.

The substrate can be pretreated with ethanol, methanol, isopropyl alcohol, nitrogen gas or air plasma to promote adhesion and remove or oxidize surface contaminants. The encapsulation device is built vertically by either moving the stage downward or the objective upward as the beam is rastered through the build volume. As described earlier, irrespective of the shape or the size of the device, the structure of the encapsulation device is fabricated to consist of a central cavity (or inner volume) with channels of varying diameter and length running through the inner volume, the channels running continuous with an outer surface or wall of the device. At the position where the channels are connected to the outer wall, a vasculature hole having a diameter corresponding to the channel is created. The central cavity is also fabricated to include a single or multiple injection ports opening through the outer wall of the device to the outside and connecting to the inner volume of the device to provide an opening through which seeding can be performed.

Once polymerized, the encapsulation device is washed with a solvent such as propylene glycol ether acetate or an alternate solvent followed by methoxy-nonafluorobutane and de-ionized water treatments to remove particulates. Still other processes or reagents may be used for particulate removal from the device. In some embodiments, the encapsulation device may be subjected to a post-baking step in which after polymerization, the device is exposed to high temperatures (e.g., in the range of 200 to 400 degrees Fahrenheit) and bright light (e.g., 200 to 500 nm wavelength light) to fully polymerize the structure. In some example embodiments, during any of the above-mentioned steps, the encapsulation device may also be sonicated with ultrasonic waves in deionized water or isopropanol to remove particulates. The encapsulation device may also be sterilized, e.g., by exposure to UV irradiation or by autoclaving.

In other example embodiments, the encapsulation device may be left attached to the substrate. For example, an encapsulation device 900 may be printed on a tether coupled to the device, such as a polyimide ribbon 910 (FIG. 9), that remains percutaneous after implantation for either easy removal, or to send electrical signals to the surface of the device, or to receive biologically generated electrical signals near or within the device site. The polyimide ribbon 910 may be fabricated to contain electronics for signal processing and transmission following implantation of the device. In other embodiments, the encapsulation device may be removed from the substrate for further processing.

In another embodiment, one or more aspects of the encapsulation device may be made of metal. As an example, the channels and/or the entire encapsulation device may be sputter coated with a metal (such as gold, titanium, stainless steel, etc.) followed by combusting the underlying polymer with a high temperature treatment (such as via exposure of the device to temperatures higher than 1000 degrees Fahrenheit). In another embodiment, metal coating may be followed by selective etching of the polymer. In all cases, a final structure of the encapsulation device may be cleaned after fabrication with a solvent and using sonication.

In another example embodiment, the encapsulation device may be coated with proteins or protein mimetics such as durable, and/or non-degradable peptoids to promote adhesion and in-growth of specific biological cells once the device is implanted. For example, extra-cellular matrix (ECM) proteins such as one or more of fibronectin, vitronectin, laminin, and collagens can be coated on all surfaces of the encapsulation device, including on the walls and all surfaces of the network of channels. In another embodiment, all surfaces of the encapsulation device may be coated with adhesion molecules such as laminin and fibronectin or growth factors such as vascular endothelial growth factor or nerve growth factor. In particular examples, the surface of the channels in fluid communication with the outer environment (e.g., a cell medium) are coated with vascular endothelial growth factors to promote vascularization of the channels.

Any known protocol for non-covalent coating of a surface may be used. One example protocol for fibronectin coating is available online at sigmaaldrich.com/technical-documents/articles/biofiles/product-protocols.html. One example protocol for laminin coating is available at sigmaaldrich.com/technical-documents/articles/biofiles/laminin-product-protocols.html. One example protocol for coating the device with VEGF and adhesion proteins is available at europepmc.org/abstract/med/27039978. One example protocol for coating the device with NGF and adhesion proteins is available at ncbi.nlm.nih.gov/pmc/articles/PMC5793558/.

The fabrication methods discussed above may also be used to provide all surfaces of the encapsulation device with a porous structure, such as via the creation of nanopores in the range of 0.02 to 3 μm pore size. Pores may be created through all wall surfaces including the inner vasculature (that is, the channels running through the inner volume of the device) during the printing process, or by using focused ion beam machining, electron beam lithography, two-photon ablation, or chemical etching. In one embodiment, the pore creation process may be combined with a nano-scale mask. Alternatively, pores larger than the resolution limit of the direct laser writing process (typically 500 nm) may be directly designed and printed along with the macrostructure.

III. Methods of Loading the Devices

Following fabrication, loading a payload into the encapsulation device may be accomplished by inserting (e.g., injecting) the payload into the inner volume of the device through the injection port.

In some embodiments, the payload is a plurality of cells. The encapsulation device is loaded by placing the device in cell culture media and injecting the cells through the injection port of the device with a micropipette and microinjector or an automatable process. In another embodiment, prior to injecting cells, the encapsulation device may be pre-filled using the same method with media such as Matrigel to promote cell health. In another embodiment, the cells may be seeded in suspension in a non-degradable PEG-based hydrogel from which active molecules can diffuse out for systemic distribution. If the cells being seeded are dormant stem cells to be activated, or stem cells to be differentiated in situ, the cells may in some instances increase in volume and lock in place. As described earlier, the payload is seeded into an inner lumen of the encapsulation device, in a region external to the channels running through an inner volume of the device.

Following payload injection into the encapsulation device, the device may be sealed. In particular, the injection port via which the payload is seeded may be closed. Various options may be used for sealing the injection port. In one embodiment, a trap door or a flap is printed over the injection port which is shut using any known sealing method, such as the door being glued shut with adhesive, or welded shut using light-induced melting. In another embodiment, the injection port may be glued shut with photoresin or with a self-curing adhesive such as silicone. In another embodiment, the injection port may be sealed by placing a plug of the same material as the encapsulation device into the hole and sealing the hole shut with light or an adhesive. At this time, the encapsulation device is ready for implanting or other uses (for example, in vitro culture of cells).

IV. Methods of Treating a Subject

Disclosed herein are example methods of treating a condition or disorder in a subject by implanting an encapsulation device loaded with a payload into a cavity or tissue of the subject. The encapsulation device is loaded with a payload that provides therapeutic molecules appropriate to treat the condition or disorder of the subject. The encapsulation device is implanted in the subject at a location that is appropriate to provide therapeutic effects for the subject's disorder. For example, if the subject has diabetes and the encapsulation device is loaded with insulin-secreting cells (such as islet cells or insulin-secreting cells), the device is implanted near the blood circulation for systemic distribution of insulin. In an embodiment where the effect is to be local and the therapeutic molecule has a short half-life, the implant can be targeted to the region or tissue to be treated.

Example methods are also provided for monitoring or managing a condition or disorder in a subject by implanting an encapsulation device loaded with a payload into the body of the subject. The encapsulation device is loaded with a payload that monitors the level of a reference molecule in a subject, the level indicative of a degree or state of the condition or disorder of the subject. For example, if the subject has an inflammatory disorder, the encapsulation device is loaded with a cell-based biosensor before implantation. Exposure of the cells of the biosensor within the encapsulation device to a cytokine specific to the inflammatory disorder may trigger a signal from the biosensor (such as trigger expression of a reporter gene of the biosensor).

Example conditions or disorders in a subject that may be treated or monitored via an encapsulation device are shown in Table 1.

TABLE 1
Exemplary disorders and payloads used for treatment
of a subject using an Encapsulation device.

| Disorder | Payload |
|---|---|
| Diabetes | Islet cells or insulin-secreting cells |
| Skin lesion | Mesenchymal stem cells |
| Duchenne muscular dystrophy | Sertoli cells |
| Thyroid | Thyroid hormone |
| Auto-immune condition | Anti-inflammatory drug |
| Pain management | Pain relief drug |
| Digestive tract inflammation | Biosensor |
| Cancer | Engineered stem cells, Erythropoietin |
| Idiopathic short stature | Human growth hormone/engineered |
| | cells |
| Turner syndrome | Human growth hormone/engineered |
| | cells |
| Chronic kidney disease | Human growth hormone, |
| | Erythropoietin/engineered cells |
| Pituitary disease | Human growth hormone/engineered |
| | cells |
| AIDS-associated anemia | Erythropoietin/C2C12 myoblasts |
| Androgen replacement therapy | Testosterone/engineered cells |
| Cell state (e.g., pH, ion | Biosensor |
| concentration, protein expression, | |
| RNA expression, etc.) | |
| Hemophilia | Erythropoietin/C2C12 myoblasts |

### EXAMPLES

The following examples are provided to illustrate certain particular features and/or embodiments. These examples should not be construed to limit the disclosure to the particular features or embodiments described.

### Example 1

### Method of Treating a Disorder Such as Diabetes in a Subject

Islet grafts have been shown to decrease or eliminate the need for insulin injections. However, due to an immune response, grafts typically do not last for more than a few years even under immune suppression. Diabetes may be treated in a subject by seeding an amount of islet cells (or insulin-secreting cells) into an injection port of the encapsulation device. This encapsulation strategy both eliminates the need for immune suppression and could conceivably last for decades.

Other use cases with similar treatment strategies include hemophilia, anti-glomerular basement membrane disease, liver disease and erythropoietin deficiency.

### Example 2

### Method of Delivering a Drug for Treating a Disorder in a Subject

The slow, targeted release of a drug can be beneficial in numerous situations including pain management, thyroid treatment, and various auto-immune disorders. An amount of drug may be seeded via an injection port of the encapsulation device. A release rate of the drug from the device may be modulated via selection of an appropriate pore size across a surface of the device. As a result, targeted doses may be tightly controlled. In addition, the high surface area surrounded by vasculature inside a lumen of the device ensures that most of the drug will directly enter the bloodstream and not create pockets that might be released unexpectedly at a later time. Targeted implantation of the device may also allow for less drug usage as the drug would already be in the region in which it was needed. In addition, local targeting can lessen off-target effects.

### Example 3

### Method of Delivering a Biosensor

A biosensor may be delivered into a lumen of the encapsulation device via the injection port. Example biosensors include digestive tract biosensors for detection of digestive tract inflammation. In one embodiment, the biosensor may include biological cells configured with a reporter gene that provide information about gastrointestinal health as the device goes through the digestive system. For example, the reporter gene may include green fluorescent protein (GFP) and exposure of the biosensor encaged within the encapsulation device to a specific inflammatory cytokine may trigger the expression of GFP.

### Example 4

### Method of Screening a Tumor Cell Line

Tumor cells of at least one tumor line may be seeded via an injection port of the encapsulation device. The device is then implanted in a subject (e.g., an animal subject used in pharmaceutical or basic research). The encapsulated tumor cells are then used to screen for compound activity in vivo against the specific tumor cell line. In another embodiment, a screen can be performed on multiple cell lines simultaneously by injecting tumor cells of multiple tumor cell lines into the device. Cells from distinct cell lines are injected into distinct devices which are then implanted and monitored for activity. This approach allows for testing and identification of the most suitable tumor line to use.

### Example 5

### Method of Treating Cancer in a Subject

Engineered stem cells delivered to a region near a tumor that are programmed to excrete specific proteins have been shown to assist in tumor cell death. For example, the release of TRAIL (secretable tumor necrosis factor apoptosis inducing ligand) has been shown to specifically target tumor cells for death. In one embodiment, stem cells engineered to excrete specific proteins, such as TRAIL or other tumor reducing factors, are seeded via an injection port of the encapsulation device and the device is then implanted at a location proximate the targeted tumor. The tumor may be malignant or benign. In another embodiment, the seeded stem cells may be engineered to excrete proteins specific for a given tumor type, tumor stage, tumor location, or tumor cell line. The approach can reduce off-target effects and increase efficacy of treatment due to cells remaining in the vicinity of the tumor after injection. The encapsulation device improves efficacy of tumor treatment by keeping the engineered stem cells in place, increasing the overall concentration of the secreted proteins.

### Example 6

### Method of Detecting a Disorder in a Subject Via a Cell-Based Biosensor

An encapsulation device may be configured to operate as a biosensor. The device may be implanted subcutaneously, within or on an organ, within a cavity of a subject, under the skull or dura, or in a large blood vessel. The device may be printed on a polyimide (FIG. 9) or silicone surface and may include a tether (such as the ribbon of FIG. 9) that includes electronics for sensing the cell state. The electronics may sense the cell state in any known way, for example, electrically, optically, based on sensed ionic concentration, pH, sensed protein levels, or sensed RNA expression levels. The sensed signal is then relayed, either with direct connection through the polyimide tether or wirelessly, to a receiver or other signal processor. The signal may be relayed, for example, through radio waves or ultrasound signal to a receiver outside the body.

### Example 7

### Method of Treating a Disorder in a Subject by Delivering a Specific Peptide or Protein

There are a number of diseases that can be treated with cells either engineered or selected for the secretion of specific peptides or proteins. In one embodiment, an encapsulation device can be seeded with one or more of the specific protein or peptide, stem cells engineered to excrete the specific protein or peptide, or biologic cells naturally secreting the specific protein or peptide. The device is then implanted at a location proximate a target location of action of the specific protein or peptide. As one example, cells engineered to secrete human growth hormone can be seeded into an encapsulation device, and the device implanted to release the growth hormone in a subject for example, to treat children with idiopathic short stature, Turner syndrome, chronic kidney disease up to the time of transplant, or in adults suffering from pituitary disease. As another example, cells engineered to secrete erythropoietin can be seeded into an encapsulation device, and the device implanted to release erythropoietin in a subject to treat chronic renal failure, cancer, or AIDs-associated anemia. In another example, engineered stem cells may be seeded into an encapsulation device to enable the release of testosterone a subject during use-cases of androgen replacement therapy.

### Example 8

### Other Uses

Various other uses of the encapsulation device of the present disclosure include implantation of grafts in plants for immune system augmentation and microbiota replacement in humans or livestock.

### Example 9

### In Vitro Uses

While the various examples described herein pertain to in vivo uses, still other in vitro uses may be possible. In one embodiment, the encapsulation device may be seeded with cells (such as stem cells) and the device may be suspended in media to enable tissue or organ generation. In another embodiment, a plurality of such encapsulation devices seeded with stem cells may be positioned relative to each other (for example, in media on a substrate, or during implantation) to allow different regions of an organ to be generated. A synergistic effect between the different regions, each independently vascularized at their corresponding device, may then allow a total organ to be created when the devices are explanted.

### Example 10

### In Vitro Cell Culture

Cells are seeded in the enclosures and kept in cell culture with or without other cells in the same culture. This is used as an assay to study drug interactions or intra and inter-cell signaling on the cells in the enclosure or cells outside the enclosure. In addition, multiple enclosures, each with its own cell type, may be cultured in the same dish, to study diffusive-molecule-only interactions between cell types (there would be no cell-cell contact interactions as the cell types are physically separated) or to increase the throughput of an assay (both by keeping cell types separate for easy sorting and by being able to test multiple cell types at once).

### Example 11

### Organ and Organoid Building Blocks

A plurality of encapsulation devices seeded with stem cells may be positioned relative to each other (for example, in media on a substrate, or during implantation) to allow different regions of a larger structure, such as an organoid or an organ to be generated. A synergistic effect between the different regions, each independently vascularized at their corresponding device, may then allow a total organ to be created when the devices are explanted. For example, the embodiment of FIG. 12 depicts multiple devices that are positioned and aligned relative to each other via the use of non-vascular structures. Such an embodiment may be used for generating different regions of an organ at each device, the different devices then positioned to align the different regions, and promote vascularization between the regions, thereby creating a whole organ.

Multiple seeded encapsulation devices may be stacked together (vertically and/or horizontally) and cultured to create either organoids (miniature, incomplete organs) or full organs. A biodegradable material may be used to create the encapsulation devices such that, as it degrades, the space will be filled with the enclosed cells' extracellular matrix proteins. Encapsulation devices can be seeded with single cell types or multiple cell types depending on the organ and where in the organ the “building block” would be placed. In order to encourage vascularization, the vascular holes in the enclosures are lined up such that fluid can flow continuously from one side of the organ(oid) to the other. To assist in alignment, indexing features are printed into the sides of the individual encapsulation devices (FIGS. 11-12). An indexing substrate may be printed on the surface of a glass coverslip or culture dish to assist initial alignment. In addition, features such as pick-points can be printed to assist in handling.

In one example, a 2D array of encapsulation devices is printed onto a planar substrate such as a glass coverslip, with fluid transport holes drilled in the glass substrate that are aligned with the cages and vasculature holes of the printed encapsulation devices. Multiple layer cell cultures are then be constructed by stacking the coverslips. To further enhance perfusion of nutrients, a microfluidic array can be attached to one or more sides of the gross structure.

Another application of the perfusion device includes endothelial cell seeding of encapsulation device vasculature in which endothelial cells are pushed through the enclosures by fluid. Adhesion proteins coating the surface of the enclosure vascular holes can catch the cells and attach them to the walls, thus creating an initial cellular wall for vasculature.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. An encapsulation device, comprising:
an outer wall defining an inner volume, the outer wall comprising a plurality of pores and a plurality of vasculature holes; and
a plurality of channels traversing the inner volume, wherein each channel extends from one of the plurality of vasculature holes to another of the plurality of vasculature holes.

2. The encapsulation device of claim 1, wherein the plurality of pores comprise openings of less than about 2 μm in diameter, the plurality of vasculature holes comprise holes of about 1 μm to about 1 mm in diameter, or both.

3. The encapsulation device of claim 1, wherein the plurality of vasculature holes comprise holes of at least two distinct diameters.

4. The encapsulation device of claim 1, wherein each channel extends from the one of the plurality of vasculature holes on a surface of the outer wall to another of the plurality of vasculature holes on an opposing surface of the outer wall.

5. The encapsulation device of claim 1, wherein the plurality of channels includes a first channel extending from a vasculature hole of a larger diameter to another vasculature hole of the same diameter, and a second channel extending from a vasculature hole of a smaller diameter to another vasculature hole of the same diameter.

6. The encapsulation device of claim 5, wherein the first channel is orthogonal to the second channel within the inner volume.

7. The encapsulation device of claim 1, wherein a diameter of a given channel of the network matches a diameter of a corresponding vasculature hole that the given channel extends to or from.

8. The encapsulation device of claim 1, wherein each of the channels comprise the plurality of pores.

9. The encapsulation device of claim 1, wherein the inner volume is divided into an inner lumen and an outer lumen by the plurality of channels, the inner lumen comprising a region of the inner volume external to the channels, the outer lumen comprising a region of the inner volume internal to the channels.

10. The encapsulation device of claim 9, wherein the outer wall further comprises at least one port selectively coupled to the inner lumen of the inner volume.

11. The encapsulation device of claim 10, wherein the port further comprises a closure mechanism.

12. The encapsulation device of claim 1, wherein the device is made of a polymer.

13. The encapsulation device of claim 1, further comprising an attachment mechanism including a seal and a recess for coupling the device to an anatomical feature following implantation into a cavity in a subject.

14. The encapsulation device of claim 1, further comprising one or more non-vascular structures for aligning one or more of the plurality of vasculature holes of the device with vasculature holes of another device or with a substrate on which the device is placed.

15. The encapsulation device of claim 1, further comprising a tether attached to the device.

16. The encapsulation device of claim 1, further comprising a payload in the inner volume of the device.

17. A method of treating a disorder in a subject, comprising:
implanting the encapsulation device of claim 16 into a tissue or cavity of the subject.

18. The method of claim 17, wherein the payload comprises a plurality of cells, a protein, a nucleic acid, an exosome, a small molecule therapeutic, or two or more thereof.

19. A method, comprising inserting a payload in the inner volume of the encapsulation device of claim 1.

20. A method of making the encapsulation device of claim 1, comprising fabricating the device using stereolithography.
